SEQUENCE LISTING (10 sequences)

SEQ ID NO: 1 - protein, 345 aa, Schistosoma haematobium
RTGYQIGKTMRLKSTSSSWNSSEAQQEMKSLYQELNNSLTSEKTFLNEKEENVVRISTGIFVQKTYEVERRFTESIANDFEGELKQVDFSQRTSATVDINDWVDQQSNGLLEKFFTDDIPDDTAMILVNVFYFRDFWQSPFEPHYTRMENFDISSDHQITVHMMTQEGVMKYGKFEDEGFEIVSKPLNNTRFTFVIVLPLEKWSLNGATELLNGNKVLSEYVKNLKETTVSLRLPKFTLKNTLDLVPTLKSIGVVDLFDPVKSDLSGITPNHNLYVNKFIQTNVFKLNESGIEATTVTSPIFVPISAIIPEVDFHVTHPFICFIYDQQLTMPIMAAKVMNPILQS

SEQ ID NO: 2 - protein, 112 aa, Schistosoma haematobium
CKYCLRLYDGTYENGSYTDVYKSVGSLSPPWIPGSVCVPLIHDSKRQPPYWRLYEDVNYSGKDTAIGHGACIDDFTKSGLKRISSIQKCVYGENGMVQCISESKRGRKYCRY

SEQ ID NO: 3 - protein, 74 aa, Schistosoma haematobium
KDRIDSEIDALMTGALDKPTPEITEFMDLIQSSFHCCGAKGPQDYGPNIPASCRGETTVYHEGCVPVFGAFLKR

SEQ ID NO: 4 - protein, 81 aa, Schistosoma haematobium
RDEVKSQFLSLVKSSVNEYSKNPDFKNFLDKIQQEFQCCGSESSSDYTSSGQTVPDSCKDTKTKAIYSDGCSYKVISFFEK

SEQ ID NO: 5 - protein, 78 aa, Schistosoma haematobium
EKPKVKKHVTDALREFVKEYSHDEHVSKVLDEVQQKLQCCGADSSKDYVTPPPESCFKDGQIFKEGCVKKVSDLSKMH

SEQ ID NO: 6 - protein, 225 aa, Schistosoma haematobium
MASLSCGYKCLQILLVIFNILVFACGIALIVIGSLSQVAINNYSSGIDSSIKGLVIFVIVLGCFLFLLGFLGFCGACTKNTCCLILYAILLSIMVAAEIAAGITAAVLRDEVKSQFLSLVKSSVNEYSKNPDFKNFLDKIQQEFQCCGSESSSDYTSSGQTVPDSCKDTKTKAIYSDGCSYKVISFFEKYIVAVLVAAFVFAILQLLCIVFAICVIRAIKSGDSD

SEQ ID NO: 7 - protein, 219 aa, Schistosoma haematobium
MALGCGYKCSQCLLVIFNCGAFICGLGLIVVGALGLHSVVNHWKDIEPPLQSLIIFIIVLGCFLFVLGALGMFGACTKNVCLLTTYCILLSILIVAEIAAGIFAILEKPKVKKHVTDALREFVKEYSHDEHVSKVLDEVQQKLQCCGADSSKDYVTPPPESCFKDGQIFKEGCVKKVSDLSKMHLNAIIISVFLFSLVQMICLVFAVCVLLAVKRGDDE

SEQ ID NO: 8 - protein, 218 aa, Schistosoma haematobium
MATLGTGMRCLKSCVFVLNIICLLCSLVLIGAGAYVEVKFSQYGDNLHKVWQAAPIAIIVVGVIILIVSFLGCCGAIKENVCMLYMYAFFLIILLIAELAAAIVAVVYKDRIDSEIDALMTGALDKPTPEITEFMDLIQSSFHCCGAKGPQDYGPNIPASCRGETTVYHEGCVPVFGAFLKRNLVIVACVAFGVCFFQLLSIVIACCLGRQIKEYENV

SEQ ID NO: 9 - protein, 372 aa, Schistosoma haematobium
GGDNFLTCPLGILFTLGILLGSGGAQGRTGYQIGKTMRLKSTSSSWNSSEAQQEMKSLYQELNNSLTSEKTFLNEKEENVVRISTGIFVQKTYEVERRFTESIANDFEGELKQVDFSQRTSATVDINDWVDQQSNGLLEKFFTDDIPDDTAMILVNVFYFRDFWQSPFEPHYTRMENFDISSDHQITVHMMTQEGVMKYGKFEDEGFEIVSKPLNNTRFTFVIVLPLEKWSLNGATELLNGNKVLSEYVKNLKETTVSLRLPKFTLKNTLDLVPTLKSIGVVDLFDPVKSDLSGITPNHNLYVNKFIQTNVFKLNESGIEATTVTSPIFVPISAIIPEVDFHVTHPFICFIYDQQLTMPIMAAKVMNPILQS

SEQ ID NO: 10 - protein, 134 aa, Schistosoma haematobium
MFLIALLSYTLINQLGITKSNPCKYCLRLYDGTYENGSYTDVYKSVGSLSPPWIPGSVCVPLIHDSKRQPPYWRLYEDVNYSGKDTAIGHGACIDDFTKSGLKRISSIQKCVYGENGMVQCISESKRGRKYCRY

(19) United States
(12) Patent Application Publication
(10) Pub. No.: US 2024/0011984 A1
(43) Pub. Date: Jan. 11, 2024

(54) PROTEINS FOR THE DETECTION OF SCHISTOSOMA INFECTION

(71) Applicant: MERCK PATENT GMBH, DARMSTADT (DE)

(72) Inventors: BEATRICE GRECO, EYSINS (CH); CLAUDE OEUVRAY, EYSINS (CH); ALEX LOUKAS, SMITHFIELD (AU); MARK PEARSON, SMITHFIELD (AU); JAVIER SOTILLO-GALLEGO, MAJADAHONDA (ES)

(21) Appl. No.: 18/033,813

(22) PCT Filed: Oct. 27, 2021
(86) PCT No.: PCT/EP2021/079886

§ 371 Date: Apr. 26, 2023

(30) Foreign Application Priority Data
Oct. 27, 2020 (EP) 20204189.3

(51) Int. Cl. G01N 33/569

(52) U.S. Cl. CPC G01N 33/569; G01N 2333/43547; G01N 2469/20

(57) ABSTRACT

The invention pertains to the field of diagnostic tools for the detection of Schistosoma infection. The invention pertains to proteins derived from Schistosoma Haematobium antigens, useful alone or in combination for the detection of anti-Schistosoma antibodies in biological samples, and thus for the diagnosis of Schistosoma infection in humans.

### FIELD OF THE INVENTION

The invention pertains to the field of diagnostic tools for the detection of Schistosoma infection. The invention pertains to proteins derived from Schistosoma haematobium antigens, useful alone or in combination for the detection of anti-Schistosoma antibodies in biological samples, and for the diagnosis of Schistosoma infection in humans.

### BACKGROUND OF THE INVENTION

Schistosomiasis, caused by infection with parasitic blood flukes of the genus Schistosoma, has a global burden of 1.86 million disability-adjusted life years. Schistosoma haematobium is the most common species affecting humans, causing urogenital schistosomiasis in approximately half of the estimated 200 million people infected throughout the world's tropical and subtropical regions (Steinmann et al. Lancet Infect Dis 2006; 6(7): 411-25). Moreover, S. haematobium infection in women substantially increases the risk of acquiring HIV/AIDS (Zirimenya et al. PLoS Negl Trop Dis 2020; 14(6): e0008383), and the International Agency for Research on Cancer (IARC) recognises urogenital schistosomiasis as a group 1 carcinogen because of its association with squamous cell carcinoma of the bladder (Moller et al. International journal of cancer Journal international du cancer 1995; 60(5): 587-9). The focus of the Schistosomiasis intervention agenda is shifting from morbidity control to eradication, and there is a WHO-mandated objective to eliminate schistosomiasis as a public health concern and interrupt transmission in selected areas. It is therefore imperative that methods to detect infection are appropriately sensitive and rapid in their execution in order to diagnose new cases of disease, assess the effectiveness of control measures and be applicable to large-scale disease surveillance.

While there is currently no gold standard recommended for detection of schistosomiasis, a widely used method for diagnosing infection involves microscopic detection of parasite eggs in urine (urogenital schistosomiasis) or faeces (intestinal schistosomiasis), which unfortunately exhibit relatively poor sensitivity in areas of low transmission due to the technique being dependent on the rate of egg excretion (Engels et al. Am J Trop Med Hyg 1996; 54(4): 319-24), limiting its value as a diagnostic tool in regions of low endemicity. However, the quantity of shed eggs depends on the parasite burden and number and intensity of freshwater contacts. Furthermore, egg-shedding varies from day to day. Tests to detect circulating schistosome antigen in the blood or urine are typically more sensitive than traditional microscopy but are not without limitations. An assay to detect circulating anodic antigen (CAA) in urine is available as a point-of-care test which has excellent capability for diagnosing moderate to high-level S. mansoni infection, but reduced performance in detecting Schistosoma haematobium infection (Midzi et al. Trans R Soc Trop Med Hyg 2009; 103(1): 45-51). Assays to detect antibodies to crude parasite preparations, such as soluble egg antigen (SEA), in urine have been shown to correlate with urine egg and serum anodic antigen levels (de Dood et al. Frontiers in immunology 2018; 9: 2635) but can have low specificity and reproducibility due to the extracts containing thousands of schistosome proteins which can cross-react with antibodies from other helminth infections. A handful of immunodiagnostics based on recombinant antigens have been developed for the medically important schistosomes (Hinz et al. Mol Cell Probes 2017; 31: 2-21). However, existing immunodiagnostics generally lack sufficient specificity to discriminate between different Schistosoma spp. In addition in the particular case of Schistosoma infections, since some infected individuals only have a low level of antibody response, either because the infection is recent or due to age-dependant antibody response, it is especially important that the immunodiagnostics achieve a great level of sensitivity.

There is thus still a need for diagnostic tools and in particular proteins or antigens that have the specificity and sensitivity necessary to diagnose Schistosoma in all individuals infected independent of the status of their infection, and that could discriminate between Schistosoma haematobium infection and infection due to other Schistosoma spp.

### SUMMARY OF THE INVENTION

The invention provides the use of at least a protein having a sequence comprising or consisting of a sequence chosen in the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 and SEQ ID NO. 10, preferably chosen in the group consisting of SEQ ID NO. 1, SEQ ID NO. 2, SEQ ID NO. 6 and SEQ ID NO. 7, for the detection of anti-Schistosoma antibodies, preferably anti-Schistosoma haematobium antibodies, in a biological sample from a human subject and/or for diagnosing a Schistosoma infection in a human subject, preferably in a biological sample from a human subject.

In other terms, the invention provides the use of at least a protein having a sequence comprising or consisting of a sequence chosen in the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 and SEQ ID NO. 10, in the manufacture of a kit for the detection of anti-Schistosoma antibodies in a biological sample from a human subject and/or for diagnosing a Schistosoma infection in a biological sample from a human subject.

The invention further provides a method for the detection of anti-Schistosoma antibodies, preferably anti-Schistosoma haematobium antibodies, in a biological sample from a human subject, said method comprising the steps of:
a. contacting said biological sample with at least one protein having a sequence comprising or consisting of a sequence chosen in the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 and SEQ ID NO. 10;
b. determining the presence and/or quantity of antibodies, in said biological sample, capable of binding to said at least one protein.

The invention also provides a method for the diagnosis of a Schistosoma infection, preferably a Schistosoma haematobium infection, in a human subject, said method comprising the steps of:
a. contacting a biological sample from the subject with at least one protein having a sequence comprising or consisting of a sequence chosen in the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 and SEQ ID NO. 10;
b. determining the quantity of antibodies, in the biological sample from the subject, capable of binding to said at least one protein.

The invention also provides a method for the treatment of a subject infected with Schistosoma, preferably Schistosoma haematobium, comprising the steps of:
diagnosing the presence or absence of Schistosoma haematobium infection in the subject according to the method of the invention,
administering the subject diagnosed with the presence of Schistosoma haematobium infection with a therapeutic treatment appropriate for Schistosoma haematobium infection.

The invention also provides a kit, preferably for the use or the method as defined herein, comprising at least a protein having a sequence comprising or consisting of a sequence chosen in the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 and SEQ ID NO. 10, and optionally a leaflet with instructions on how to use said at least one protein.

### Definitions

By “protein” “polypeptide” and “peptide” it is herein referred to a molecule comprising amino acids joined via peptide bonds. In general, “peptide” is used to refer to a sequence of 20 or less amino acids and “polypeptide” is used to refer to a sequence of greater than 50 amino acids. However, in the present application, for the sake of clarity and ease of reading, the “proteins”, “polypeptides” and “peptides” of the invention are all referred to as “proteins”. The proteins, polypeptides and peptides according to the invention may be purified, produced by a recombinant process (i.e., expression of exogenous nucleic acid encoding the peptide, polypeptide or protein in an organism, host cell, or cell-free system) or produced by chemical synthesis.

By “sensitivity”, in reference to a test, assay or diagnosis, it is herein referred to the test's ability to detect the proportion of true positive subjects with the disease in a total group of subjects with the disease. Hence, it relates to the potential of a test to identify subjects with the disease.

By “specificity”, in reference to a test, assay or diagnosis, it is herein referred to the test's ability to correctly detect the proportion of subjects without the disease with negative test result in total of subjects without disease. In other words, specificity represents the probability of a negative test result in a subject without the disease. Therefore, specificity relates to the aspect of diagnostic accuracy that describes the test ability to identify subjects without the disease, i.e. to exclude the condition of interest.

By “antibody” or “antibodies”, it is herein generally referred to immunoglobulins. When referring to antibodies which presence and/or quantity is to be determined according to the invention, the term “antibodies” refer to naturally occurring antibodies, that is to say antibodies that would be present in the biological sample from the subject as a product of the subject's immune response, as opposed to antibodies which would have been added to the biological sample and/or would have been injected to the patient. In this context, the term antibodies refer to antibodies of the IgA, IgD, IgE, IgG and IgM isotype. Preferably in the context of the invention, the antibodies which presence and/or quantity is to be determined according to the invention are of the IgG isotype. In the context of the invention, the term “anti-Schistosoma antibodies” refer to antibodies naturally occurring antibodies capable of binding to Schistosoma antigens.

When referring to antibodies used as biological tools in an assay or test, for instance as secondary and/or detection antibodies, the term “antibodies” refer to any type of antibody that would be fit for that purpose, independent of the species of origin or isotype.

Sequences

SEQ ID NO. 1 corresponds to the minimal useful sequence from MS3_013701

SEQ ID NO. 2 corresponds to the minimal useful sequence from Sh-TSP-2

SEQ ID NO. 3 corresponds to the minimal useful sequence from MS3_09198

SEQ ID NO. 4 corresponds to the minimal useful sequence from MS3_10385

SEQ ID NO. 5 corresponds to the minimal useful sequence from MS3_10186

SEQ ID NO. 6 corresponds to the sequence of MS3_013701

SEQ ID NO. 7 corresponds to the sequence of Sh-TSP-2

SEQ ID NO. 8 corresponds to the sequence of MS3_09198

SEQ ID NO. 9 corresponds to the sequence of MS3_10385

SEQ ID NO. 10 corresponds to the sequence of MS3_10186

### BRIEF DESCRIPTION OF THE FIGURES

FIG. 1. Serum and urine IgG responses resulting from probing of S. haematobium protein arrays. (A) Volcano plot showing fold change and significance of IgG responses between infected (S. haematobium-endemic) and non-infected populations with serum or (B) urine. Each individual spot denotes a single arrayed antigen. Dotted lines represent different probability thresholds (from bottom up: first line, p<0.05; second line, p<0.01; third line, p<0.001). (C) Scatterplot showing correlation of serum and urine IgG responses for all samples (serum, n=242; urine, n=117) and (D) matched serum and urine samples (n=17).

FIG. 2. IgG antibody responses to E. coli-expressed recombinant versions of top-ranked proteins and S. haematobium soluble egg antigen (Sh-SEA) in sera from S. haematobium-endemic populations. (A) anti-MS3_10385; (B) anti-MS3_10186; (C) anti-MS3_09198; (D) anti-MS3_01370; (E) anti-Sh-TSP-2; (F) anti-Sh-SEA. Egg-positive subjects were characterized (WHO stratification) as either having a high (≥50 eggs per 10 ml urine) or low (1-49 eggs per 10 ml urine) intensity infection. “egg −ve/CAA +ve”=egg negative subjects who are classified as positive (infected) by the more sensitive circulating anodic antigen (CAA) detection test. “egg −ve/CAA −ve”=egg negative subjects who are confirmed as antigen negative by the CAA detection test. “non-end. −ve”=subjects from a non-endemic area. Plotted data represent the responses of both the Zimbabwe and Gabon cohorts. Reactivity cutoffs were determined as the average plus 3SD of the values of the non-endemic negative group (dotted line). Significance of the difference in antibody levels between each infected group and the non-infected group were analyzed by Student's t test *p≤0.05, **p≤0.01, ***p≤0.001, ****p≤0.0001.

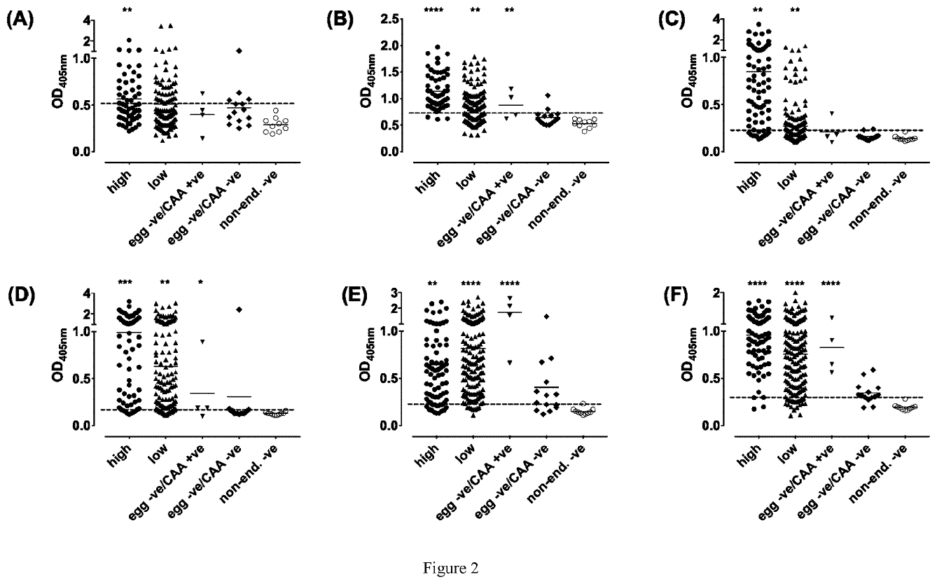

FIG. 3. IgG antibody responses to E. coli-expressed recombinant versions of top-ranked proteins and S. haematobium soluble egg antigen (Sh-SEA) in urine from S. haematobium-endemic populations. (A) anti-MS3_10385; (B) anti-MS3_10186; (C) anti-MS3_09198; (D) anti-MS3_01370; (E) anti-Sh-TSP-2; (F) anti-Sh-SEA. Egg-positive subjects were characterized (WHO stratification) as either having a high (≥50 eggs per 10 ml urine) or low (1-49 eggs per 10 ml urine) intensity infection. “egg −ve/CAA +ve” =egg negative subjects who are classified as positive (infected) by the more sensitive circulating anodic antigen (CAA) detection test. “egg −ve/CAA −ve”=egg negative subjects who are confirmed as antigen negative by the CAA detection test. “non-end. −ve”=subjects from a non-endemic area. Plotted data represent the responses of both the Zimbabwe and Zanzibar cohorts. Reactivity cutoffs were determined as the average plus 3SD of the values of the non-endemic negative group (dotted line). Significance of the difference in antibody levels between each infected group and the non-infected group were analyzed by Student's t test *p≤0.05, **p≤0.01, ***p≤0.001, ****p≤0.0001.

FIG. 4. Results of pilot development of a Point of Care Immunochromatographic Test (PoC-ICT) for diagnosis of urogenital schistosomiasis using serum. Schematic showing positive (dark boxes) and negative (white boxes) results for MS3_01370 or Sh-TSP-2 ICT using ELISA-validated serum samples from the Gabon cohort (high, n=10; low, n=22; egg −ve/CAA +ve, n=4; egg −ve/CAA −ve, n=14; non-end. −ve, n=10). Samples have been sorted from left to right by decreasing egg burden and FoR percentages among the infected populations (sensitivity) are displayed on the right-hand side of the image. Brief description of the PoC-ICT: strips were coated with either MS3_01370 or Sh-TSP-2 at the test line to facilitate capture and detection of anti-MS3_01370 or anti-Sh-TSP-2 IgG in serum added to the sample reservoir. Appearance of a band at the test and control lines was considered a positive result and a band at the control line only was considered a negative result. Test bands were given a score from least (+1) to most (+4) intense and a score of 0 was given to a negative result. Two independent readers had to agree on a test result. Every test performed was valid, as confirmed by the appearance of a band at the control line.

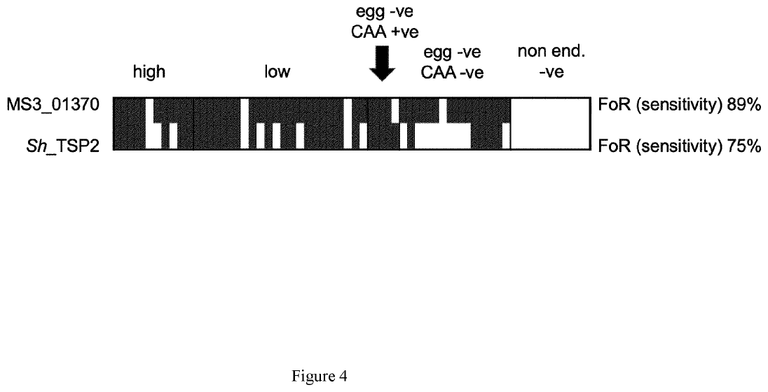

FIG. 5. Scatterplot showing correlation of S. haematobium infection intensity (urine egg burden) with individual mean SI of all spots resulting from probing of S. haematobium protein arrays with different diagnostic fluids. (A) Serum. (B) Urine.

FIG. 6. Receiver operating characteristic curve showing diagnostic performance (area under the curve—AUC) of the minimal antibody signature in each diagnostic fluid. (A) Serum. (B) Urine.

FIG. 7. Antibody responses to combinations of cell-based recombinant versions of top-ranked proteins generated by ELISA with human urine from S. haematobium-endemic populations. (A) anti-MS3_10385+anti-MS3_10186+anti-MS3_09198 IgG response. (B) anti-MS3_10385+anti-MS3_09198+anti-MS3_01370+anti-Sh-TSP-2 IgG response. (C) anti-MS3_09198+anti-MS3_01370+anti Sh-TSP-2 IgG response. (D) anti-MS3_01370 IgG response+anti-Sh-TSP-2 IgG response. Egg-positive subjects were characterized (WHO stratification) as either having a high (≥50 eggs per 10 ml urine) or low (1-49 eggs per 10 ml urine) infection. “egg −ve/CAA +ve”=egg negative subjects who are classified as positive (infected) by the more sensitive circulating anodic antigen (CAA) detection test. “egg −ve/CAA −ve”=egg negative subjects who are confirmed as antigen negative by the CAA detection test. “non-end. −ve”=subjects from a non-endemic area. Plotted data represent the responses of both the Zimbabwe and Zanzibar cohorts. Reactivity cutoffs were determined as the average plus 3SD of the values of the non-endemic negative group (dotted line). Significance of responses between each infected group and the non-infected group were analysed by Student's t test *p≤0.05, **p≤0.01, ***p≤0.001, ****p≤0.0001.

FIG. 8. Frequency of recognition (FoR) patterns based on IgG ELISA responses to combinations of cell-based recombinant versions of top-ranked proteins and Sh-SEA using urine from infected individuals in S. haematobium-endemic populations. Samples have been sorted from left to right by decreasing egg burden. For each antigen, dark bars represent recognition by a sample from the Zimbabwe cohortor by a sample from the Zanzibar cohort and white bars denote no recognition (below the cutoff determined by ELISA), regardless of cohort. “Combination 1”=MS3_10385+MS3_10186+MS3_09198, “combination 2”=MS3_10385+MS3_09198+MS3_01370+Sh-TSP-2, “combination 3”=MS3_09198+MS3_01370+Sh-TSP-2, “combination 4”=MS3_01370+Sh-TSP-2. FoR percentages among the infected populations (sensitivity) are displayed on the right-hand side of the pattern. “Zim”=Zimbabwe cohort, “Zan”=Zanzibar cohort, “all”=samples from both cohorts combined. To facilitate proper comparison, the dataset has been trimmed to exclude any samples not assayed for all four recombinant antigen combinations (n=148).

FIG. 9. Serum IgG ELISAs showing recognition of S. haematobium diagnostic antigens by other schistosome species. “Sh”=S. haematobium-infected serum samples from the Gabon cohort used previously in this study. “Sm”=S. mansoni-infected serum samples. “Sj”=S. japonicum-infected serum samples. “non-end. −ve”=serum samples from a non-endemic area used previously in this study.

### DETAILED DESCRIPTION OF THE INVENTION

The invention is based in part on the discovery of proteins useful for the detection of antibodies against Schistosoma haematobium and/or for the diagnosis of Schistosoma infection in biological samples such as serum or urine from human subjects. The inventors have demonstrated, as illustrated in the experimental part, that some specific proteins can be used efficiently to detect antibodies, in particular IgGs, produced by the subject as part of the host immune response upon Schistosoma haematobium infection, using biological samples such as urine or serum. Each of these proteins, which are part of the Schistosoma haematobium transcriptome, are recognized with great sensitivity and specificity by infected host's antibodies, and enable the diagnosis of S. haematobium infection even in patient which have a low number of parasite eggs in their urine, hence facilitating the detection of new cases when the infection is still at an early stage. In addition, these proteins have a higher specificity for antibodies occurring in the host, in particular in humans, as a result of S. haematobium infection than for antibodies occurring as a result of infections due to other Schistosoma species. The proteins disclosed herein, as well as the uses, methods and kits based thereof, enable a specific and early diagnosis particularly useful for the practitioner in that it can be used to design an efficient treatment strategy.

The invention therefore pertains to proteins which can be used alone or in combination as antigens to detect the presence of antibodies directed to S. haematobium in a biological sample from a subject, and can be used to diagnose S. haematobium infection. The invention provides methods and kits that can be useful to implement the invention and may be used as a preliminary step prior to any therapeutic intervention such as for instance therapeutic treatments for S. haematobium infection or an infection associated therewith. The invention provides the use of at least a protein having a sequence comprising or consisting of a sequence chosen in the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 and SEQ ID NO. 10, preferably chosen in the group consisting of SEQ ID NO. 1, SEQ ID NO. 2, SEQ ID NO. 6 and SEQ ID NO. 7, for the detection of anti-Schistosoma antibodies, preferably anti-Schistosoma haematobium antibodies, in a biological sample from a human subject and/or for diagnosing a Schistosoma infection in a human subject, preferably in a biological sample from a human subject.

In other terms, the invention provides the use of at least a protein having a sequence comprising or consisting of a sequence chosen in the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 and SEQ ID NO. 10, in the manufacture of a kit for the detection of anti-Schistosoma antibodies in a biological sample from a human subject and/or for diagnosing a Schistosoma infection in a biological sample from a human subject.

The invention further provides a method for the detection of anti-Schistosoma antibodies, preferably anti-Schistosoma haematobium antibodies, in a biological sample from a human subject, said method comprising the steps of:
c. contacting said biological sample with at least one protein having a sequence comprising or consisting of a sequence chosen in the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 and SEQ ID NO. 10;
d. determining the presence and/or quantity of antibodies, in said biological sample, capable of binding to said at least one protein.

Preferably, the at least one protein is an isolated or recombinant protein, more preferably a recombinant protein. By “recombinant protein” it is herein referred to a protein produced by genetic engineering and/or recombinant technology. Briefly, for expressing a protein by recombinant technology, a protein-encoding nucleotide sequence is placed in operable connection with a promoter or other regulatory sequence capable of regulating expression in an expression system, such as a suitable cell-free system or cellular system, and the expression system is placed for a time and under conditions sufficient for expression to occur. Alternatively, isolated proteins may be prepared using solid-phase synthesis, such as that generally described by Merrifield, J. Amer. Chem. Soc, 85:2149-54 (1963), although other equivalent chemical syntheses known in the art may also be used. Solid-phase peptide synthesis may be initiated from the C-terminus (or N-terminus) of the peptide by coupling a protected amino acid to a suitable resin.

Preferably, the at least one protein has a sequence comprising or consisting of, preferably consisting of, a sequence chosen in the group consisting of SEQ ID NO. 1 SEQ ID NO. 1, SEQ ID NO. 2, SEQ ID NO. 6 and SEQ ID NO. 7. More preferably, the at least one protein has a sequence comprising or consisting of, preferably consisting of, a sequence chosen in the group consisting of SEQ ID NO. 1 and SEQ ID NO. 6.

Advantageously, to increase specificity and sensibility of the detection and/or diagnosis, in the use and/or the method of the invention two or more of the proteins as defined herein are used. Preferably, in those embodiments, at least two proteins having a sequence comprising or consisting of a sequence chosen in the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 and SEQ ID NO. 10, are used. Yet preferably, in those embodiments, at least a protein having a sequence comprising or consisting of sequence SEQ ID NO. 1 or of sequence SEQ ID NO. 6, and a protein having a sequence comprising or consisting of sequence SEQ ID NO. 2 or of sequence SEQ ID NO. 7, are used.

The sequence or structure of the proteins of the invention may be modified to facilitate their use in the methods and kits of the invention, for instance by the addition of a N-terminal or C-terminal sequence, or the conjugation to molecules of interest. Advantageously, the proteins of the invention comprise the recited sequence and further comprise an additional N-terminal peptide sequence and/or an additional C-terminal sequence. In such embodiments, the additional N-terminal peptide sequence and/or additional C-terminal sequence preferably comprises or consists of 1, 2, 3, 4, 5, 6, 7, 8, 9, 10, or 20 amino acids. Advantageously, the proteins of the invention are conjugated to a ligand or carrier protein, such as for instance biotin, avidin, streptavidin, neutravidin or serum albumin.

By “biological sample”, it is herein referred to a sample obtained from a human subject. A biological sample may comprise tissues and/or biological fluids. Such samples can be obtained in vitro, ex vivo or in vivo. As a non-limiting example, the biological sample may be selected from tissues, organs, cells, or any isolated fraction of a human subject. The biological sample may also be selected from blood, plasma, lymph, saliva, urine, stool, tears, sweat, sperm, or cerebrospinal fluid, synovial, pleural, peritoneal, or pericardial, and any fraction or extracts thereof. Preferably, the biological sample is a biological fluid that comprises IgG antibodies. As is known in the art, in humans, IgG antibodies can be found in blood, serum, saliva, urine, lymph fluid, cerebrospinal fluid and peritoneal fluid. More preferably, the biological sample is blood, plasma, serum and urine. Even more preferably, the biological sample is chosen from the list consisting of serum and urine. Yet preferably, the biological sample is serum.

Said sample can be obtained by any technique known in the art. The biological sample may be pre-processed to preserve the integrity of the antibodies of interests and/or to make them more accessible for further analysis. The biological sample may for instance undergo centrifugation, purification, or other treatment steps to facilitate access to antibodies, in particular IgGs, and/or to concentrate them. The biological sample may also be pre-processed so as to limit or lower the presence of antibodies, in particular IgGs, susceptible to react in a non-specific way with the proteins of the invention. The biological sample may for instance undergo a pre-absorption step, wherein non-specific antibodies reacting against the biological species in which the proteins of the invention are recombinantly produced are removed from or blocked in the biological sample. Such a pre-absorption step may be implemented by contacting the biological sample with a sample or extract from the organism in which the proteins according to the invention are being produced. For instance, the biological sample may be pre-adsorbed for anti-E. coli antibodies by being contacted with E. coli lysate, in particular when the proteins of the invention are recombinant proteins produced in E. coli.

Preferably, in the method according to the invention, the presence and/or quantity of antibodies refers to the presence and/or quantity of IgG antibodies.

By “capable of binding”, used in reference to antibodies, it is herein referred to antibodies capable of binding to defined proteins under the usual experimental conditions of immunogenic assays. Preferably, the antibodies are capable of binding specifically to the at least one protein as defined herein. In the context of the invention, an antibody which “binds specifically” to a defined protein forms or undergoes a physical association with it, in an amount and for a time to sufficient allow detection of the antibody-protein complex. By “specifically” or “preferentially,” it is meant that the antibody has a higher affinity for defined protein than for other proteins, such as for instance other proteins contained in the biological sample. In the context of the invention, the term “affinity” when referring to antibodies, designate the strength with which said antibody binds to a defined protein, or a part thereof, and is measured by the affinity constant between the antibody and its antigen (defined as 1/K_{D}, wherein K_{D} is the dissociation constant as classically defined) measurement of the reaction rate constants can be used to define an equilibrium or affinity constant (I/K_{D}). The affinity of an antibody for its target is thus inversely correlated to the dissociation constant, i.e. the smaller the K_{D} value the greater the affinity of the antibody for its target. For example, the antibody can have an affinity for the defined protein of at least about 1.5-fold, 2-fold, 2.5-fold, 3-fold, or higher than for other proteins in the sample. Such affinity or degree of specificity can be determined by a variety of routine procedures, including competitive binding assays. It should be understood that in the context of the invention, the proteins of the invention have been selected for the ability to bind specifically to anti-Schistosoma antibodies, that is to say the proteins of the invention have a greater affinity for anti-Schistosoma antibodies, and in particular to anti-Schistosoma haematobium antibodies, than for other antibodies present in human biological samples.

By “determining the presence and/or quantity”, when in reference to the antibodies present in the biological sample from the subject, it is herein referred to qualitative or quantitative determination of the presence of the specific antibodies under investigation. Phrases such as “sample comprising an antibody” or “determining the presence and/or quantity of an antibody in a sample” are not meant to exclude samples or determinations where no antibody is contained or detected. In a general sense, this invention involves assays to determine whether an antibody as part of the host's immune response to infection with Schistosoma, in particular S. haematobium is present in a sample, and therefore does not pretend to preclude situations wherein no such antibody is present or detected is the sample. In the context of the invention, the presence and/or quantity of antibodies capable of binding to at least a protein as defined in the invention can be determined using any appropriate technique known in the field, including but not limited to protein detection chips, bead-based assays, lateral flow devices, and enzyme-linked immunosorbent assays (ELISA). Preferably, the presence and/or quantity of antibodies in the biological sample is assayed by immunoassay, more preferably antigen-based immunoassay. Such assays typically allow for the quantitative detection of protein/antibody complex formation, and further, the experimental conditions can easily be set so as to ensure that protein/antibody complex formation being detected result from specific binding between the at least one protein according to the invention and the antibodies which presence is to be determined.

In the context of the invention, the term “immunoassay” should be construed as generally understood in the art, that is to say as referring to an assay that is meant to detect or measure an analyte based on the interaction between an immunological reagent, usually an antibody, and its ligand. For the sake of clarity, the terms “antigen-based immunoassay” herein refers to an immunoassay wherein one or several antigens are used as reagents to detect and/or quantify the analyte which is an antibody. For the sake of clarity, the terms “antibody-based immunoassay” refers to an immunoassay wherein one or several antibodies are used as reagents to detect and/or quantify the analyte which is an antigen. Typically, in the case of an antigen-based immunoassay, at least one antigen of interest is immobilized on a solid support, and the sample to be tested is brought into direct contact under conditions such that any specific antibodies in the sample bind to the immobilized antigen. If such specific antibodies capable of binding to the antigen of interest are present in the sample, a complex is formed, which presence and/or quantity can be detected either by direct or indirect means, such as for instance by secondary antibodies.

For instance, standard solid phase ELISA and lateral flow immunoassay are quantitative immunoassays which can be used to perform either antigen-based immunoassays or antibody-based immunoassays and are particularly useful in determining the quantity or concentration of a protein or antibody from a variety of patient samples. These techniques are well known, and have been described thoroughly in the literature, for instance antigen-specific enzyme-linked immunosorbent assay (antigen-specific ELISA), was described by Engvall et al. (in The Journal of Immunology. 109 (1): 129-135 (1972)) and lateral flow immunoassays, also called lateral flow tests, were described for instance by Koszula and Gallotta (in Essays Biochem. 60(1):111-20 (2016)).

Preferably, the presence and/or quantity of antibodies is assayed by ELISA or lateral flow immunoassay. Preferably, determining the presence and/or the quantity of antibodies capable of binding to at least a protein as defined in the invention comprises a step of detecting the formation of a complex formed between the at least one protein of the invention and antibodies, preferably IgGs, present in the biological sample.

Preferably, detecting the formation of a complex formed between the at least one protein of the invention and antibodies, preferably IgGs, present in the biological sample involves the use of secondary antibodies, also called detection antibodies. In the context of the invention, the term “secondary antibodies” refers to antibodies capable of binding to the anti-Schistosoma antibodies present in the biological sample. It will be immediately apparent that anti-human IgG antibodies can be used as secondary antibodies and enable the detection of the complex formed between the at least one of the invention and the IgG anti-Schisostoma antibodies present in the sample to be assayed. In the context of the invention, the terms “anti-human IgG antibodies” refers to antibodies specific of human IgG antibodies, that is to say antibodies capable of binding to human IgG antibodies with a greater affinity than their affinity to other human immunoglobulins. Such anti-human IgG antibodies are typically used as secondary antibodies in immunoassays, their production and use are well known in the art and they are commercially available. In the context of the invention, said anti-human IgG antibodies can be monoclonal or polyclonal, and can originate from any species other than the species of the subject (i.e. human), such as mouse, rat or goat. As is typically performed in the art, the anti-human IgG antibodies can be conjugated to a detectable label. Preferably, the secondary antibodies are anti-human antibodies, preferably anti-human IgG antibodies.

Preferably, in the method of the invention, the presence and/or quantity of antibodies is assayed by immunoassays, preferably by ELISA or lateral flow immunoassay, and involves the use of anti-human IgG antibodies, preferably anti-human IgG antibodies conjugated to a detectable label.

By “detectable label” it is herein referred to a molecule or composition bound to an analyte, analyte analog, detector reagent, or binding partner that is detectable by spectroscopic, photochemical, biochemical, immunochemical, electrical, optical or chemical means. Examples of labels, including enzymes, colloidal gold particles, colored latex particles, have been disclosed for instance in U.S. Pat. Nos. 4,275,149, 4,313,734, 4,373,932 and 4,954,452. Additional examples of useful labels include, without limitation, radioactive isotopes, co-factors, ligands, chemiluminescent or fluorescent agents, protein-adsorbed silver particles, protein-adsorbed iron particles, protein-adsorbed copper particles, protein-adsorbed selenium particles, protein-adsorbed sulphur particles, protein-adsorbed tellurium particles, protein-adsorbed carbon particles, and protein-coupled dye sacs. The attachment of a compound (e.g., a detector reagent) to a label can be through covalent bonds, adsorption processes, hydrophobic and/or electrostatic bonds, as in chelates and the like, or combinations of these bonds and interactions and/or may involve a linking group. Preferably, the detectable label is chosen in the list consisting of a fluorophore, chemiluminescent and radioactive label. Examples of suitable labels include colloidal gold, a fluorophore (such as for instance a fluorescent dye such as FITC or Texas Red, a fluorescent protein such as GFP, or a nanocrystal such as Qdot probes) or an enzyme (such as for instance horseradish peroxidase (HRP)), alkaline phosphatase (AP) or β-galactosidase), all commonly used in immunoassays.

In the context of the invention, and as is common practice in the field, the person skilled in the art may set the conditions of the immunoassay so as to guaranty or improve the selectivity and specificity of the immunoassay, for instance by using proteins non-specific of anti-Schisostoma antibodies, such as for instance Bovine Serum Albumin (BSA), and putting such proteins in contact with the sample to be assayed. In the context of the invention, proteins non-specific of anti-Schisostoma antibodies refer to proteins unlikely to bind specifically to anti-Schisostoma antibodies, for instance protein from species other than from the Schisostoma genus, preferably proteins originating from species other than trematodes.

In the context of the invention, it should be understood that the step of determining the presence and/or quantity of anti-Schistosoma antibodies may include a step of acquiring data resulting from the assay, and a further step of expressing and/or computing said data into information representing the presence and/or quantity of anti-Schistosoma antibodies. For instance, determining the presence and/or quantity of anti-Schistosoma antibodies may include a step of acquiring data from the immunoassay, such as for example data relative to the level of detectable label detected in the sample after completion of the immunoassay, and optionally further determining from this data the presence and/or quantity of anti-Schistosoma antibodies in the biological sample, for example by comparing said data to a reference value or a set of reference values. The reference value or the set of reference values may be a predetermined value or set of values, such as a predetermined standard value or set of values established for calibration purposes.

As thoroughly detailed in the experimental part, it has been established by the inventors that the presence and/or quantity of anti-Schistosoma IgGs in a biological sample from a human subject correlates with the diagnosis of Schistosoma, in particular Schistosoma haematobium infection in said subject. Moreover, the proteins as herein disclosed, and used in the method for the detection of anti-Schistosoma antibodies of the invention, have a greater specificity for anti-Schistosoma haematobium IgGs than for human IgGs naturally occurring in biological samples as a result from the infection of the host by other species of Schistosoma.

It will be immediately apparent that the method for the detection of anti-Schistosoma antibodies as described herein can be useful in the diagnosis a Schistosoma infection, in particular a Schistosoma haematobium infection, and moreover can advantageously be used to distinguish a Schistosoma haematobium infection from a Schistosoma mansoni or Schistosoma japonicum infection in a human subject.

The invention also provides a method for the diagnosis of a Schistosoma infection, preferably a Schistosoma haematobium infection, in a human subject, said method comprising the steps of.
c. contacting a biological sample from the subject with at least one protein having a sequence comprising or consisting of a sequence chosen in the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 and SEQ ID NO. 10;
d. determining the quantity of antibodies, in the biological sample from the subject, capable of binding to said at least one protein.

The features as defined herein in relation to the method for the detection of anti-Schistosoma antibodies as described herein apply to the method for the diagnosis of a Schistosoma infection. Preferably, the method for the detection of anti-Schistosoma antibodies is an in vitro method.

Preferably, the method for the diagnosis of a Schistosoma infection according to the invention further comprises a step c) of comparing the quantity determined in step b) with the quantity of antibodies, capable of binding to said at least one protein, present in a reference sample.

In the context of the invention, the quantity of antibodies, capable of binding to said at least one protein, present in a reference sample may either be predetermined threshold values, or be determined in the course of implementing the method of the invention as an active step of said method. Thus, in an embodiment, the method for the diagnosis of a Schistosoma infection according to the invention comprises a step c) of determining the quantity of antibodies capable of binding to said at least one protein present in a reference sample, and further a step d) of comparing the quantity of antibodies as determined in step b) with the quantity of antibodies as determined in step c). Preferably, the reference sample is derived from biological samples from one or more subjects not infected with Schistosoma, preferably from one or more subjects not infected with Schistosoma haematobium. In another embodiment, the method for the diagnosis of a Schistosoma infection according to the invention comprises a step of comparing the quantity as determined in step b) with a reference threshold value. In the context of the invention, the reference threshold value is preferably the quantity of antibodies capable of binding to said at least one protein, present in a reference sample, wherein the reference sample is derived from biological samples from one or more subjects not infected with Schistosoma, preferably from one or more subjects not infected with Schistosoma haematobium.

Preferably, the method for the diagnosis of a Schistosoma infection according to the invention further comprises a further step of diagnosing from the comparison of step c) or from the comparison of step d) the presence or the absence of Schistosoma infection in the subject. Preferably, in said step of diagnosing
a quantity determined in step b) superior to a quantity of antibodies as determined in step c) or to a reference threshold value indicates the presence of Schistosoma infection in the subject, and
a quantity determined in step b) inferior or equal to a quantity of antibodies as determined in step c) or to a reference threshold value indicates the absence of Schistosoma infection in the subject.

More preferably, in the method for the diagnosis of a Schistosoma infection:
the quantity of antibodies as determined in step c) or the reference threshold value is the quantity of antibodies capable of binding to said at least one protein, present in a reference sample, wherein the reference sample is derived from biological samples from one or more subjects not infected with Schistosoma haematobium,
and in said step of diagnosing:
a quantity determined in step b) superior to a quantity of antibodies as determined in step c) or to a reference threshold value indicates the presence of Schistosoma haematobium infection in the subject, and
a quantity determined in step b) inferior or equal to a quantity of antibodies as determined in step c) or to a reference threshold value indicates the absence of Schistosoma haematobium infection in the subject.

It is noteworthy that the efficacy of available schistosomiasis treatments varies greatly depending on the Schistosoma species responsible for the infection. For instance, although praziquantel and oxamniquine are considered equivalent in relation to efficacy against Schistosoma mansoni, oxaminiquine has been shown to lack efficacy against the urogenital form of the disease caused by Schistosoma haematobium. The method for the diagnosis of a Schistosoma haematobium infection according to the invention may be used as a preliminary step prior to therapeutic intervention, in order to design or adapt the therapeutic treatment.

The invention thus also provides a method for the treatment of a subject infected with Schistosoma, preferably Schistosoma haematobium, comprising the steps of:
diagnosing the presence or absence of Schistosoma haematobium infection in the subject according to the method of the invention,
administering the subject diagnosed with the presence of Schistosoma haematobium infection with a therapeutic treatment appropriate for Schistosoma haematobium infection.

In the context of the invention, therapeutic treatments appropriate for Schistosoma haematobium infection include praziquantel, metrifonate, artesunate or mefloquine. Preferably, the therapeutic treatment appropriate for Schistosoma haematobium infection is chosen in the list consisting of praziquantel, metrifonate, and the combination of praziquantel and either metrifonate, artesunate or mefloquine. Yet preferably the therapeutic treatment appropriate for Schistosoma haematobium infection comprises or consists of praziquantel.

The invention also provides a kit, preferably for the use or the method as defined herein, comprising at least a protein having a sequence comprising or consisting of a sequence chosen in the group consisting of SEQ ID NO. 1, SEQ ID NO. 2, SEQ ID NO. 3, SEQ ID NO. 4, SEQ ID NO. 5, SEQ ID NO. 6, SEQ ID NO. 7, SEQ ID NO. 8, SEQ ID NO. 9 and SEQ ID NO. 10, and optionally a leaflet with instructions on how to use said at least one protein.

Preferably, the kit according to the invention further comprises anti-human IgG antibodies conjugated to a detectable label.

Preferably, in the kit according to the invention, said at least a protein is immobilized on a solid support.

By “solid support” it is herein referred to material which is insoluble, or can be made insoluble by a subsequent reaction. Numerous and varied solid supports are known to those in the art and include, without limitation, nitrocellulose, the walls of wells of a reaction tray, multi-well plates, test tubes, polystyrene beads, magnetic beads, membranes, microparticles (such as latex particles), and sheep (or other animal) red blood cells. Any suitable porous material with sufficient porosity to allow access by detector reagents and a suitable surface affinity to immobilize capture reagents is contemplated by this term. For example, the porous structure of nitrocellulose has excellent absorption and adsorption qualities for a wide variety of reagents, for instance, capture reagents. Nylon possesses similar characteristics and is also suitable. Microporous structures are useful, as are materials with gel structure in the hydrated state.

Further examples of useful solid supports include: natural polymeric carbohydrates and their synthetically modified, cross-linked or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers which may be prepared with suitably porous structures, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; porous inorganic materials such as sulfates or carbonates of alkaline earth metals and magnesium, including barium sulfate, calcium sulfate, calcium carbonate, silicates of alkali and alkaline earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer.

The protein may be immobilized on the solid support by ionic interactions, hydrophobic interactions, covalent linkage or by adsorbing on to any substrate known in the art such as for instance poly-lysine. The surface of a solid support may be activated by chemical processes that cause covalent linkage of an agent (e.g., a capture reagent) to the support. Except as otherwise physically constrained, a solid support may be used in any suitable shapes, such as films, sheets, strips, or plates, or it may be coated onto or bonded or laminated to appropriate inert carriers, such as paper, glass, plastic films, or fabrics.

The scope of the invention will also be apparent from the following description of specific examples.

### Examples

Materials and Methods

Study Design and Cohorts

All samples used in this study were leveraged from previous studies with ethical approval and had been stratified based on egg burden as determined by microscopy analysis of urine samples (high, ≥50 eggs per 10 ml urine; or light, 1-49 eggs per 10 ml urine). Egg-negative samples were further tested for the presence of circulating anodic antigen (CAA) using the up-converting phosphor lateral flow CAA assay (de Dood et al. Frontiers in immunology 2018; 9: 2635) and were classified as being egg negative/CAA positive or egg negative/CAA negative. Protein arrays were probed with serum and urine samples from S. haematobium-endemic regions of Zimbabwe and Gabon. ELISAs were performed using the same cohorts of serum samples from Zimbabwe and Gabon and the same cohort of urine samples from Zimbabwe only (the Gabon urine cohort had been exhausted from array probing). Additional ELISA validation was performed using urine samples from an elimination setting: Zanzibar, United Republic of Tanzania. Species specificity analysis was performed using Schistosoma japonicum-infected samples from The Philippines, and Schistosoma mansoni-infected samples from Ethiopia. ICT evaluation was performed with serum samples from the Gabon cohort (table 1).

TABLE 1
Serum and urine sample cohorts used in the study.

| | | | | | Egg burden | | | | | | | | | |
|---|---|---|---|---|---|---|---|---|---|---|---|---|---|---|
| | | | | | | | | | Egg | Egg | | | | |
| | | | | | | | | | negative/ | negative/ | | | | |
| | | Cohort | Cohort age | | | | | | CAA | CAA | Study | | | |
| | | size | Range | Mean | | Heavy^{&} | Moderate ^{#} | Light\hat{ } | positive | negative | component* | | | |
| Region | Sample | (n) | (Years) | (Years) | Range | (n [%]) | (n [%]) | (n [%]) | (n [%]) | (n [%]) | A | E | ICT | SS |
| Mashonaland East | serum | 201 | 5-73 | 9.8 | 0.3-743 | 64 | 53 | 84 | 0 | 0 | ✓ | ✓ | | |
| province, Zimbabwe | | | | | | [31.8] | [26.4] | [41.8] | [0.0] | [0.0] | | | | |
| Lambarene, Gabon | serum | 56 | 8-63 | 24.0 | 0-1265 | 13 | 11 | 14 | 4 | 14 | ✓ | ✓ | ✓ | ✓ |
| | | | | | | [23.2] | [19.6] | [25.0] | [7.1] | [25.0] | | | | |
| Mashonaland East | urine | 97 | 7-15 | 10.6 | 0-1008 | 31 | 25 | 34 | 6 | 1 | ✓ | ✓ | | |
| province, Zimbabwe | | | | | | [32.0] | [25.8] | [35.0] | [6.2] | [1.0] | | | | |
| Lambarene, Gabon | urine | 27 | 8-18 | 12.0 | 0-1265 | 5 | 7 | 5 | 0 | 10 | ✓ | | | |
| | | | | | | [18.5] | [25.9] | [18.5] | [0.0] | [37.0] | | | | |
| Pemba/Unjuga | urine | 152 | 9-39 | 11.4 | 0-540 | 7 | 15 | 45 | 4 | 81 | | ✓ | | |
| islands, Zanzibar | | | | | | [4.6] | [9.9] | [29.6] | [2.6] | [53.3] | | | | |
| Northern Samar, | serum | 21 | 7-22 | 12.16 | 24-5616 | 134 | 54 | 42 | 0 | 0 | | | | ✓ |
| The Philippines | | | | | | [58.3] | [23.5] | [18.3] | [0.0] | [0.0] | | | | |
| Gondar, Ethiopia | serum | 230 | | | 10-650 | 3 | 8 | 10 | 0 | 0 | | | | ✓ |
| | | | | | | [14.3] | [38.1] | [43.5] | [0.0] | [0.0] | | | | |

*A = array, E = ELISA, ICT = immunochromatographic test, SS = species specificity.
^{&}For S. haematobium infection, heavy egg burden ≥50 eggs/10 ml urine; for S. mansoni and S. japonicum infection, heavy egg burden >400 eggs per gram faeces.
^{#} For S. haematobium infection, moderate egg burden = 11-49 eggs/10 ml urine; for S. mansoni and S. japonicum infection, moderate egg burden = 101-400 eggs per gram faeces.
\hat{ }For S. haematobium infection, light egg burden = 1-10 eggs/10 ml urine; for S. mansoni and S. japonicum infection, light egg burden = 1-100 eggs per gram faeces.

Probing of S. haematobium Protein Arrays with Human Sera and Urine

Serum IgG responses to arrayed antigens were determined by probing with human serum (1:50 in array blocking buffer/10% E. coli lysate) as previously described (Ochodo et al. Cochrane Database Syst Rev 2015; (3): CD009579) with the exception that an anti-human IgG-Qdot conjugate (1:100 in array blocking buffer) was used as the secondary/detection antibody and so tertiary incubation with a separate detection reagent was not needed. Urine IgG responses were determined by probing arrays in the same way except that human urine samples were first concentrated 15-fold and buffer-exchanged into PBS before being diluted 1:5 in array blocking buffer/10% E. coli lysate and applied to the arrays.

S. haematobium Protein Array Construction, Probing and Analysis

Proteins present in the adult S. haematobium tegument, soluble excretory/secretory products (ES) and EVs, as well as ES from the egg stage (Sotillo et al. PLoS Negl Trop Dis 2019; 13(5): e0007362), and S. haematobium orthologues of the S. mansoni schistosomula tegument proteome¹⁷ were selected for printing on the array. The remaining proteins consisted of S. haematobium orthologues of select proteins from the S. mansoni proteome array (. Array construction was the same as described previously except that open reading frames of arrayed proteins were commercially synthesised, instead of amplifying every gene from parasite cDNA using PCR. Parasite extracts were included on the array as positive controls.

Serum IgG responses to arrayed antigens were determined by probing with human sera (1:50 in array blocking buffer/10% E. coli lysate) as previously described (Gaze et al. PLoS Pathog 2014; 10(3): e1004033) with the exception that an anti-human IgG-Qdot conjugate (1:100 in array blocking buffer) was used as the secondary/detection antibody. Urine IgG responses were determined by probing arrays in the same way except that human urine samples were first concentrated 15-fold and buffer-exchanged into PBS before being diluted 1:5 in array blocking buffer/10% E. coli lysate.

S. haematobium Protein Array Data Analysis and Bioinformatics

Datasets generated from probing arrays with serum and urine were analysed separately. The signal intensity (SI) of each spot was background corrected and transformed using the variance stabilizing normalization (vsn) method in GMine using the VSN Bioconductor package (Proietti et al. Sci Rep 2016; 6: 38178). Antibody responses were considered reactive if the mean SI for all infected individuals for that protein was greater than the mean plus 1.5 standard deviations (SD) of the SI of all non-endemic negatives. Significant differences between antibody responses in the infected versus non-infected groups were determined by Student's t-test. Receiver-operating characteristic (ROC) curves and area under curve (AUC) values were generated for each antibody response using the ROCR R package and the protein targets of those responses were ranked in order of response significance.

A set of antigens producing an antibody signature which could most effectively discriminate between infected and non-infected individuals, using either serum or urine as the diagnostic fluid, was identified. Firstly, for each dataset, all antigens which were the targets of an antibody response that was significantly higher in the infected compared to non-infected populations were selected. From these, antigen targets of responses with a frequency of positivity (reactivity) in less than 30% of the infected population and more than 30% of the non-infected population were also excluded. Antigens in these trimmed datasets were sorted by greatest to least fold change in mean SI between the infected and non-infected populations and frequency of reactivity in the infected population. The top 5 antigens in each dataset were used to build a support vector machine classifier, the performance of which was evaluated by Monte Carlo cross-validation (Proietti et al. Sci Rep 2016; 6: 38178).

Antibody Signature Identification

A set of antigens capable of producing an antibody signature which could most effectively discriminate between infected and non-infected individuals, using either serum or urine as the diagnostic fluid, was identified. Firstly, for each dataset, all antigens inducing an antibody response that was significantly upregulated (after correction for multiple testing) between the infected and non-infected populations were selected (serum, n=208; urine, n=45). From these, antigens inducing antibody responses with a frequency of positivity (reactivity) in less than 30% of the infected population (serum, n=178; urine, n=7) and more than 30% of the non-infected population (serum, n=9; urine, n=13) were also excluded. Antigens in these trimmed datasets (serum, n=21; urine, n=25) were sorted by greatest to least fold change in mean SI between the infected and non-infected populations and frequency of reactivity in the infected population. The top 5 antigens in each dataset were used to build a support vector machine classifier, the performance of which was evaluated by Monte Carlo cross-validation. Iteratively, 15 samples were randomly selected as the training set and the remaining samples were used as the test set.

The model was then fit to the training data, and the predictive accuracy of the model in classifying samples as infected or non-infected was assessed using the testing set. This process was repeated 4 times. The predictive performance of the model was then evaluated by averaging the ROC curves across all 4 Monte Carlo cross-validation runs.

Selection of EV-Derived TSPs

TSPs present in the S. haematobium EV proteome were sorted by abundance (protein spectrum counting), and the most abundant TSPs with homologues of diagnostic efficacy reported in the literature (n=3) were selected for further assessment.

Recombinant Protein Production in E. coli

Eight antigens, selected from the immune signature and EV proteomic set (MS3_10385, MS3_10186, MS3_06193, MS3_01466, MS3_05950, MS3_09198, MS3_013701 and Sh-TSP-2), were expressed in E. coli as previously described (Pearson et al. PLoS Negl Trop Dis 2012; 6(X): e1564). Expression yields of MS3_06193, MS3_01466 and MS3_05950 were at levels too low to warrant further development.

ELISA Validation of Serum and Urine IgG Responses

IgG responses to E. coli-expressed and purified recombinant proteins in each biofluid were measured by ELISA. Plates (Greiner) were coated with antigen, blocked and probed with sera (1:50) followed by goat anti-human IgG-HRP (Sigma, 1:5000), and developed with TMB. Urine IgG responses to each antigen were measured in a similar way except that urine samples were diluted 1:10. Urine IgG responses to multiple antigens were performed in the same way and plates were coated with antigen diluted to 2 μg/ml. Species specificity analysis was performed as for serum ELISAs. Assays were performed in triplicate and blank-corrected values were plotted using Graphpad Prism 7. Reactivity cutoffs were determined as the mean plus 3SD of the non-endemic negative group. ROC curves were generated using Graphpad Prism 7.

Pilot Development of PoC-ICTs

For PoC test development, a lateral flow ICT was designed (Serve Science, Bangkok, Thailand). The conjugate pad was coated with 10 OD of gold-conjugated mouse anti-human IgG, either recombinant (1.0 mg/ml) MS3_01370.1 or Sh-TSP-2 was sprayed at the test line and 1.0 mg/ml anti-mouse IgG was sprayed at the control line. Serum (5 μl, diluted 1:10 in buffer BS-007) was applied to the sample reservoir, 3 drops of buffer BS-007 was applied to the sample reservoir and the test was read after 15 mins. For each strip, bands at the test and control lines was a positive result, a band at the control line only was a negative result and a test was invalid if there was no band at the control line. Band intensity on positive tests was scored on a four-point scale from most (+4) to least (+1) intense. A score of 0 was given for a negative result. Test results were confirmed by two independent and blinded examiners.

Results

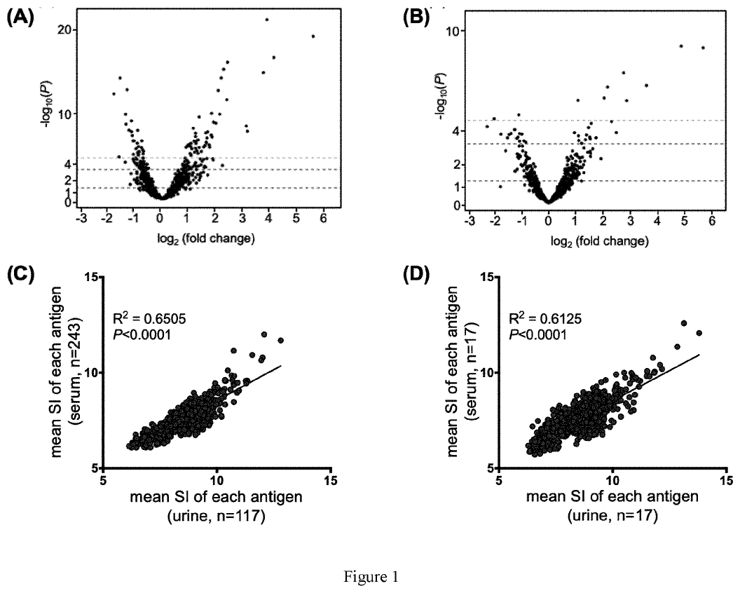

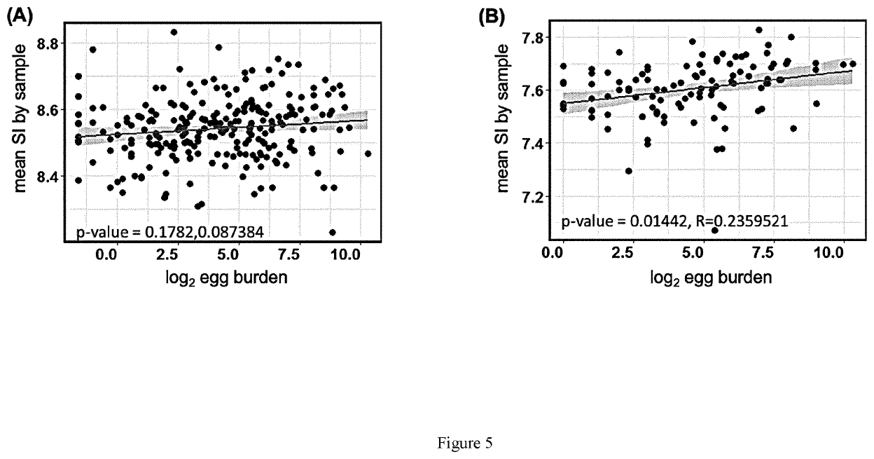

Serum and urine IgG responses to numerous arrayed antigens were significantly elevated in the infected versus non-infected population (serum, n=208; urine, n=45) (FIGS. 1A and B). The antigens producing the top 20 most significant responses are listed (table 2), with the majority of these detected in proteomic studies and, of these, at least half in each dataset were identified from the EV fraction of the parasite proteome. Seven of these top 20 antigens (35%) were shared between the two datasets. There was a significant correlation between serum and urine IgG responses (R²=0.651, p<0.0001) from all samples used to probe the arrays (serum, n=243; urine, n=117), supported by a significant correlation between the responses from a subset of matched (n=17) serum and urine samples (R²=0.613, p<0.0001) (FIGS. 1C and D). For urine, IgG SI significantly correlated with infection intensity (R²=0.234, p<0.05) but there was no correlation between infection intensity and serum IgG SI (R²=0.087, p>0.05) (FIG. 5).

TABLE 2
Top 20 antigens with significantly higher IgG responses in the
serum and urine of infected versus non-infected individuals.

| WBPS14^{$} | | | Selection method for |
|---|---|---|---|
| Accession | Description^{#} | P value* | array inclusion |
| Serum | | | |
| MS3_10186.1 | IPSE^{#} | 2.5 × 10⁻²⁰ | bioinformatic |
| MS3_10385.1 | neuroserpin^{#} | 1.6 × 10⁻¹⁷ | proteomic (T, ES, EV) |
| MS3_02553.1 | saposin containing protein^{#} | 3.8 × 10⁻¹⁵ | proteomic (T, EV) |
| MS3_09207.1 | hemoglobinase (C13 family)^{#} | 1.0 × 10⁻¹⁴ | proteomic (EV) |
| MS3_09198.1 | CD63 antigen^{#} | 5.8 × 10⁻¹⁴ | proteomic (T, EV) |
| MS3_07972.1 | ferritin, heavy polypeptide 1 | 1.2 × 10⁻¹³ | proteomic (ES, EV) |
| MS3_01370.1 | CD63 antigen | 3.8 × 10⁻¹³ | proteomic (T, EV) |
| MS3_05950.1 | 16 kDa calcium-binding protein^{#} | 1.0 × 10⁻¹¹ | proteomic (T, ES, EES) |
| MS3_06828.1 | calcium-binding mitochondrial carrier protein SCaMC-1 | 1.0 × 10⁻¹⁰ | proteomic (T) |
| MS3_01658.1 | phospholipase D3 | 4.2 × 10⁻⁹ | proteomic (ES, EV) |
| MS3_06368.1 | 5′-AMP-activated protein kinase subunit beta-1 | 4.7 × 10⁻⁹ | bioinformatic |
| MS3_07892.1 | guanine nucleotide-binding protein subunit beta | 9.5 × 10⁻⁹ | proteomic (T) |
| MS3_01466.1 | band 7 protein | 3.5 × 10⁻⁸ | bioinformatic |
| MS3_00180.1 | putative programmed cell death protein | 4.4 × 10⁻⁸ | proteomic (T, EV) |
| MS3_08105.1 | peptidyl-prolyl cis-trans isomerase B | 5.7 × 10⁻⁸ | bioinformatic |
| MS3_06193.1 | PUR-alpha-like protein^{#} | 8.5 × 10⁻⁸ | proteomic (ES) |
| MS3_09175.1 | aquaporin-3 (AQP-3) | 1.5 × 10⁻⁷ | proteomic (T, EV) |
| MS3_09779.1 | cathepsin B-like peptidase (C01 family) | 3.0 × 10⁻⁷ | proteomic (EV) |
| MS3_09828.1 | Rho GDP-dissociation inhibitor 2 | 3.3 × 10⁻⁷ | bioinformatic |
| MS3_10252.1 | steroid dehydrogenase, putative | 4.3 × 10⁻⁷ | bioinformatic |
| Urine | | | |
| MS3_10385.1 | neuroserpin^{#} | 4.2 × 10⁻¹¹ | proteomic (T, ES, EV) |
| MS3_06193.1 | PUR-alpha-like protein^{#} | 1.8 × 10⁻⁷ | proteomic (ES) |
| MS3_10186.1 | IPSE/alpha^{#} | 1.8 × 10⁻⁷ | bioinformatic |
| MS3_02553.1 | saposin containing protein^{#} | 4.3 × 10⁻⁶ | proteomic (T, EV) |
| MS3_01466.1 | band 7 protein | 3.5 × 10⁻⁸ | bioinformatic |
| MS3_07702.1 | troponin T | 2.0 × 10⁻⁵ | proteomic (T, ES, EV) |
| MS3_04688.1 | zinc finger CDGSH domain-containing protein 1 | 7.7 × 10⁻⁵ | proteomic (T) |
| MS3_00996.1 | sh3 domain grb2-like protein B1 (endophilin B1) | 8.6 × 10⁻⁵ | proteomic (T, ES, EV) |
| MS3_09198.1 | CD63 antigen^{#} | 8.6 × 10⁻⁵ | proteomic (T, EV) |
| MS3_07481.1 | fimbrin, putative | 1.2 × 10⁻³ | proteomic (T, ES, EV) |
| MS3_09207.1 | hemoglobinase (C13 family)^{#} | 1.3 × 10⁻³ | proteomic (EV) |
| MS3_07178.1 | putative ferritin | 2.3 × 10⁻³ | proteomic (T, ES, EES, EV) |
| MS3_05950.1 | 16 kDa calcium-binding protein^{#} | 3.6 × 10⁻³ | proteomic (T, ES, EES) |
| MS3_02428.1 | cytochrome b-c1 complex subunit 7 | 3.6 × 10⁻³ | proteomic (T) |
| MS3_00180.1 | putative programmed cell death protein | 3.7 × 10⁻³ | proteomic (T, EV) |
| MS3_01257.1 | BC026374 protein (S09 family) | 4.1 × 10⁻³ | bioinformatic |
| MS3_01857.1 | paramyosin isoform 1 | 4.5 × 10⁻³ | Proteomic (EES) |
| MS3_04820.1 | putative zinc transporter | 4.5 × 10⁻³ | bioinformatic |
| MS3_03509.1 | protein jagged-1b | 4.9 × 10⁻³ | bioinformatic |
| MS3_09779.1 | cathepsin B-like peptidase (C01 family)^{#} | 5.1 × 10⁻³ | proteomic (EV) |

^{$}WormBase ParaSite online database, version 14
^{#}hits common to serum and urine.
*corrected for multiple testing.
T = adult tegument, ES = adult excretory/secretory products, EES = egg excretory/secretory products, EV = adult extracellular vesicles.
****for feature selection, ref proteomic to Sotillo J, Pearson M S, Becker L, et al. In-depth proteomic characterization of Schistosoma haematobium: Towards the development of new tools for elimination. PLoS Negl Trop Dis 2019; 13(5): e0007362, Sh ELV proteome ms or both; ref bioinformatic to de Assis R R, Ludolf F, Nakajima R, et al. A next-generation proteome array for Schistosoma mansoni. Int J Parasitol 2016; 46(7): 411-5

TABLE 3
Subset of arrayed antigens inducing an antibody signature^{#} able to most effectively
discriminate between infected and non-infected populations using sera or urine.

| | | Serum IgG response | | | Urine IgG response | | |
|---|---|---|---|---|---|---|---|
| WBPS14^{$} | | Fold | Infected | AUC^{&} | Fold | Infected | AUC^{&} |
| accession | Description | change\hat{ } | (%)* | (95% CI) | change\hat{ } | (%)* | (95% CI) |
| MS3_10385.1 | neuroserpin⁺ | 47.47 | 81.07 | 0.88 (0.83-0.92) | 90.02 | 91.15 | 0.93 (0.85-1.00) |
| MS3_10186.1 | IPSE | 14.59 | 95.47 | 0.88 (0.83-0.92) | 50.96 | 87.61 | 0.88 (0.80-0.97) |
| MS3_06193.1 | PUR-alpha-like protein | 8.58 | 53.09 | 0.71 (0.64-0.79) | 29.67 | 79.65 | 0.83 (0.75-0.91) |
| MS3_01466.1 | band 7 protein | 3.71 | 66.26 | 0.69 (0.62-0.75) | 12.18 | 52.21 | 0.75 (0-66-0.84) |
| MS3_05950.1 | 16 kDa calcium-binding | 4.18 | 69.55 | 0.76 (0.70-0.82) | 5.64 | 56.64 | 0.72 (0.62-0.82) |
| | protein | | | | | | |
| MS3_09198-1 | CD63 antigen⁺ | 4.81 | 95.88 | 0.79 (0.73-0-85) | 7.39 | 91.15 | 0.83 (0-73-0-92) |
| MS3_09779.1 | cathepsin B-like peptidase | 8.85 | 32.92 | 0.84 (0.76-0.93) | 2.56 | 26.55 | 0.66 (0-55-0.76) |
| | (C01 family)⁺ | | | | | | |
| MS3_07972.1 | ferritin, heavy polypeptide 1⁺ | 13.33 | 55.97 | 0.86 (0.80-0.92) | 2.36 | 45.13 | 0.65 (0.54-0.76) |
| MS3_09207.1 | hemoglobinase (C13 family)⁺ | 5.31 | 30.45 | 0.78 (0.71-0.84) | 2.97 | 34.51 | 0.68 (0.57-0.78) |
| MS3_01370.1 | CD63 antigen⁺ | 4.53 | 32.51 | 0.78 (0.71-0.84) | 2.23 | 38.05 | 0.66 (0.56-0.77) |

^{#}A minimum of four antigens is predicted to achieve the most effective discrimination between infected and non-infected populations using sera or urine.
^{$}WormBase ParaSite online database, version 14
⁺Antigens were identified from proteomic analysis of S. haematobium EVs.
\hat{ }mean of the log₂-transformed fold change of the signal intensity between the infected and non-infected population
*Percentage of infected population samples (positive by egg microscopy or CAA) that are positive by array probing.
^{&}Area under the curve (AUC) value determined from receiver-operator characteristic (ROC) curve analysis of the IgG response to each antigen.

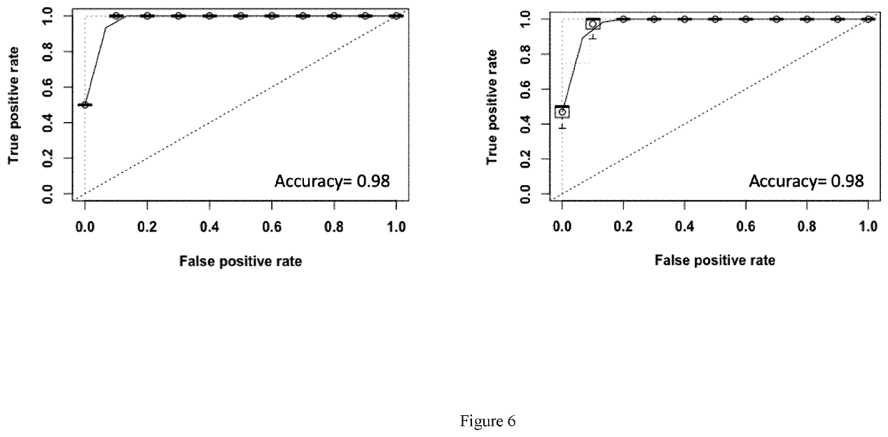

An antibody signature was identified which could most effectively discriminate between infected and non-infected populations by using either serum or urine as the diagnostic fluid (table 3). From this set, it was determined that a minimum of four antigens were capable of producing an antibody signature with a diagnostic accuracy (AUC) of 0.98 in either diagnostic sample (FIG. 6). Antigens that were the targets of this response included IPSE (MS3_10186), serpin (MS3_10385), two CD63-like TSPs (MS3_09198 and MS3_01370) and a 16 kDa calcium-binding protein (MS3_05950). The majority of these antigens were identified from the S. haematobium EV proteome.

To validate the diagnostic performance of antigens identified from protein array probing, five antigens from either the immune signature or EV proteomic set (MS3_10385, MS3_10186, MS3_09198, MS3_01370 and Sh-TSP-2) were used to measure IgG responses by ELISA. Urine IgG responses were further assessed using samples from a region of low transmission for S. haematobium (Knopp et al. Lancet Glob Health 2019; 7(8): e1118-e29). Serum antibody responses to all recombinant antigens were significantly reactive in all infected cohorts, except for MS3_10186.1 and MS3_09198.1, where only responses to the high and medium infection intensity groups reached significance. Of the recombinant antigens, the antibody response that was most significantly reactive in the egg negative/CAA positive group (the cohort with the lowest level of infection) was to Sh-TSP-2. Of the antibody responses to purified recombinant antigens, those with the greatest ability to discriminate between the infected and non-infected populations (both Zimbabwe and Gabon cohorts combined) were against MS3_0371 and Sh-TSP-2, generating AUCs of 0.93 and 0.97, respectively (table 4). A frequency of recognition (FoR) pattern analysis among the infected populations revealed that, consistent with the high AUC values of these molecules, MS3_0170 and Sh-TSP-2 were the two recombinant antigens most frequently recognized and, especially in the case of Sh-TSP-2, this was due to greater recognition by individuals with a lower infection intensity (data not shown). Specificity for all recombinant antigens was 100% in all cohorts tested due to the stringent reactivity cutoff set (mean+3SD of all non-endemic negative samples) for all assays.

TABLE 4
Diagnostic accuracy of antigens using serum of individuals from
Schistosoma haematobium-endemic populations determined by ELISA.

| | AUC in cohort (95% CI) | | |
|---|---|---|---|
| Antigen | Zimbabwe | Gabon | All |
| MS3_10385 | 0.79 (0-68-0.91) | 0.83 (0.71-0.95) | 0.80 (0.70-0.91) |
| MS3_10186 | 0.85 (0-78-0.92) | 0.98 (0.94-1.00) | 0.88 (0.82-0.94) |
| MS3_09198 | 0.82 (0.73-0.91) | 0.86 (0.75-0.96) | 0.82 (0.74-0.91) |
| MS3_01370 | 0.93 (0.88-0.98) | 0.95 (0.88-1.00) | 0.93 (0.89-0.97) |
| Sh-TSP2 | 0.97 (0.94-1.00) | 1.00 (1.00-1.00) | 0.98 (0.95-1.00) |
| Sh-SEA | 0.97 (0.94-0.99) | 1.00 (1.00-1.00) | 0.97 (0.95-1.00) |

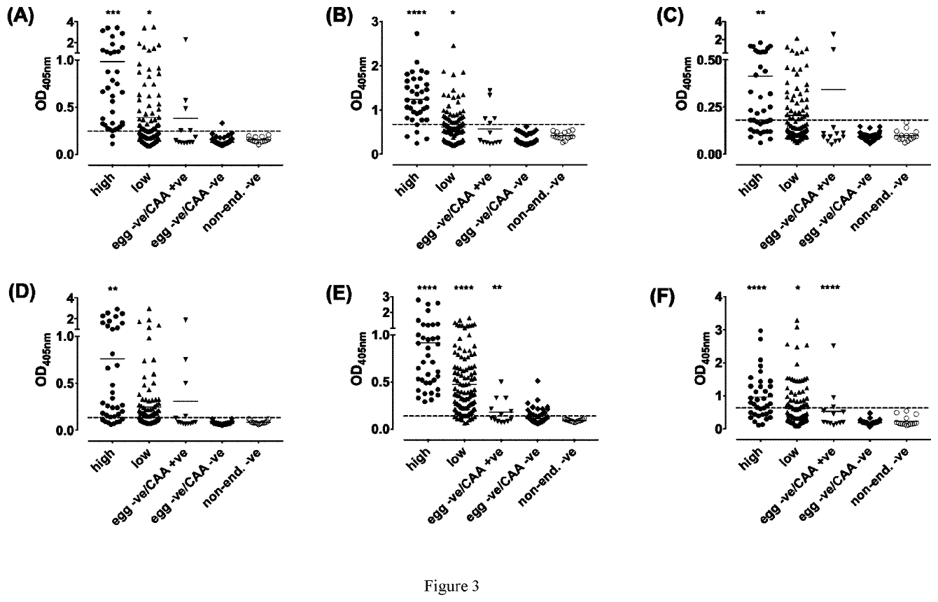

Urine IgG responses to all recombinant antigens in the high and medium infection intensity groups were significantly reactive compared to controls. Additionally, anti-Sh-TSP-2 responses in both the low and egg negative/CAA positive infection intensity groups were also significantly elevated compared to controls (FIG. 3). AUC values for the antibody responses to all recombinant antigens were high (>0.89) in the Zimbabwe cohort and were modest in the cohort from the elimination setting in Zanzibar (0.57-0.69) for all recombinant antigens except Sh-TSP-2 (0.93) (table 5).

TABLE 5
Diagnostic accuracy of antigens using urine of individuals from
Schistosoma haematobium-endemic populations determined by ELISA.

| | AUC in cohort (95% CI) | | |
|---|---|---|---|
| Antigen | Zimbabwe | Zanzibar | All |
| MS3_10385 | 0.95 (0.90-0.99) | 0.57 (0.46-0.69) | 0.78 (0.71-0.86) |
| MS3_10186 | 0.96 (0.92-1.00) | 0.66 (0.55-0.77) | 0.69 (0.62-0.77) |
| MS3_09198 | 0.94 (0.90-0.98) | 0.66 (0.51-0.81) | 0.78 (0.67-0.88) |
| MS3_01370 | 0.89 (0.82-0.96) | 0.69 (0.56-0.82) | 0.81 (0.72-0.89) |
| Sh-TSP2 | 0.98 (0.96-1.00) | 0.93 (0.87-0.99) | 0.96 (0.93-0.99) |
| Sh-SEA | 0.77 (0.68-0.87) | 0.82 (0.68-0.95) | 0.79 (0.69-0.90) |

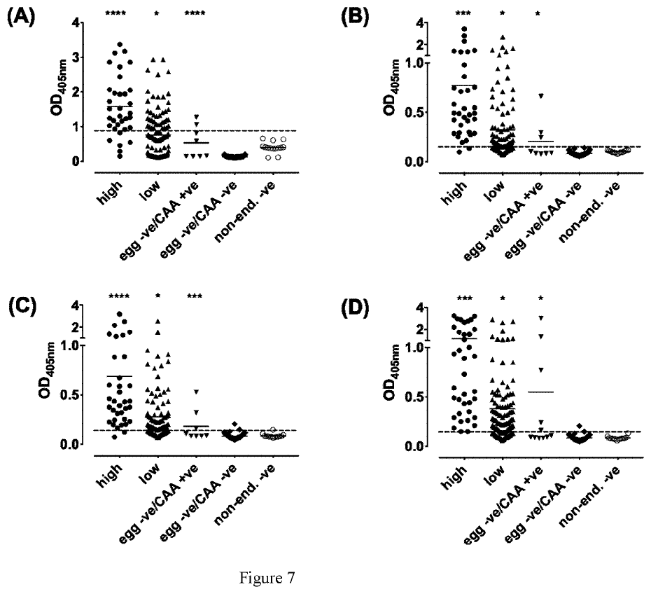

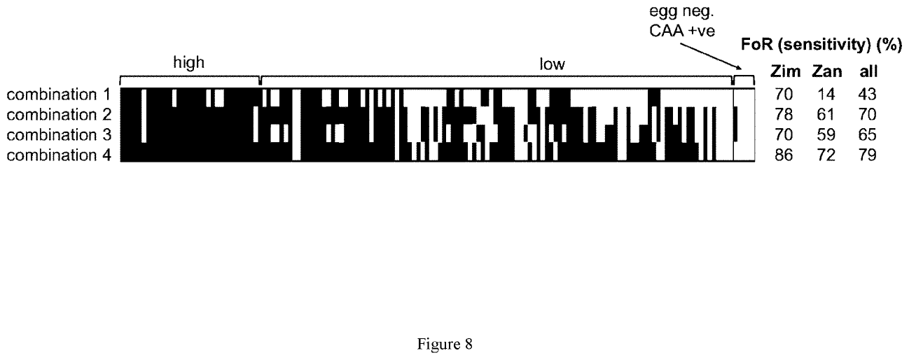

For urine, the high diagnostic performance of Sh-TSP-2 was reflected in the FoR pattern analysis, which shows, as for the serum cohorts, the recognition of this antigen, above all others tested, in the low infection intensity group (data not shown). Given the differences in FoR patterns of the molecules tested, various combinations of the antigens were tested to see if these cocktails would elicit a higher positivity rate among the infected population and increase diagnostic performance. All antigen combinations resulted in significant responses from all infected cohorts, compared to controls (FIG. 7). The use of these combinations, however, did not result in an increase of AUC or FoR value in the infected population compared to Sh-TSP-2 alone (table 6, FIG. 8). As with the serum ELISAs, specificity for all recombinant antigens in all cohorts tested was absolute.

TABLE 6
Diagnostic accuracy of antigen combinations using
urine of individuals from Schistosoma haematobium-endemic
populations determined by ELISA.

| Antigen | AUC in cohort (95% CI) | | |
|---|---|---|---|
| combination | Zimbabwe | Zanzibar | All |
| MS3_10385 + | 0.94 (0.89-0.98) | 0.63 (0.48-0.80) | 0.70 (0.61-0.79) |
| MS3_10186 + | | | |
| MS3_ 09198 | | | |
| MS3_10385 + | 0.93 (0.87-0.98) | 0.79 (0.70-0.88) | 0.88 (0.83-0.93) |
| MS3_09198 + | | | |
| MS3_01370 + | | | |
| Sh-TSP2 | | | |
| MS3_09198 + | 0.94 (0.89-1.00) | 0.92 (0.85-0.99) | 0.93 (0.87-0.98) |
| MS3_01370 + | | | |
| Sh-TSP2 | | | |
| MS3_01370 + | 0.97 (0.93-1.00) | 0.92 (0.86-0.98) | 0.95 (0.91-0.99) |
| Sh-TSP2 | | | |

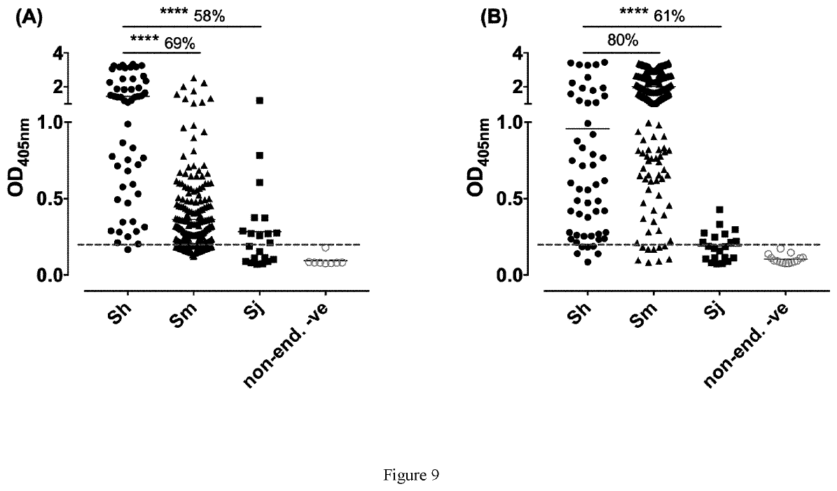

The extent of recognition of Sh-TSP-2 and MS3_01370 (the two highest-performing antigens) in sera from individuals mono-infected with either S. mansoni or S. japonicum. Both antigens were recognized to a significantly lesser degree by serum antibodies from S. japonicum infections and, in the case of Sh-TSP-2, S. mansoni infections (FIG. 9).

Finally, whether the diagnostic performance of Sh-TSP-2 and MS3_01370 could be translated into a field-compatible format was assessed. A PoC-ICT was designed with each antigen coated at the test line to capture and detect their cognate antibodies present in the sera of infected people (the ICT design had been optimized for use with this fluid as recommended by the manufacturer). ICTs coated with either Sh-TSP-2 or MS3_01370 detected antibodies at every level of infection intensity from the cohort sampled (89% and 75% sensitivity, respectively), even in individuals who were egg negative by urine filtration and were only positive for CAA. Both sets of ICTs displayed 100% specificity, returning negative results for every non-endemic sample tested.

1-13. (canceled)

14. A method for the detection of anti-Schistosoma antibodies in a biological sample from a human subject, said method comprising the steps of:
a) contacting said biological sample with at least one protein having a sequence comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10; and
b) determining the presence and/or quantity of antibodies, in said biological sample capable of binding to said at least one protein.

15. The method according to claim 14, said method comprising the steps of:
a) contacting a biological sample from the subject with at least one protein having a sequence comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10; and
b) determining the quantity of antibodies, in the biological sample from the subject, capable of binding to said at least one protein.

16. The method according to claim 14, wherein the anti-Schistosoma antibodies are anti-Schistosoma haematobium antibodies.

17. The method according to claim 14, wherein the at least one protein has a sequence comprising SEQ ID NO: 1 SEQ ID NO: 1, SEQ ID NO: 2, SEQ ID NO: 6 or SEQ ID NO: 7.

18. The method according to claim 14, wherein the biological sample contains at least two proteins having a sequence comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10.

19. The method according to claim 14, wherein the biological sample contains:
a protein having a sequence comprising SEQ ID NO: 1 or SEQ ID NO: 6, and
a protein having a sequence comprising SEQ ID NO: 2 or SEQ ID NO: 7.

20. The method according to claim 15, wherein the method further comprises a step c) of determining the quantity of antibodies capable of binding to said at least one protein present in a reference sample, and further a step d) of comparing the quantity of antibodies as determined in step b) with the quantity of antibodies as determined in step c).

21. The method according to claim 15, wherein the method further comprises a step of comparing the quantity as determined in step b) with a reference threshold value.

22. A kit comprising at least one protein having a sequence comprising SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9 or SEQ ID NO: 10, and optionally a leaflet with instructions on how to use said at least one protein.

23. The kit according to claim 22, wherein said at least one protein is immobilized on a solid support.

24. The kit according to claim 22, wherein the kit further comprises anti-human IgG antibodies conjugated to a detectable label.
